(19) United States
(12) Patent Application Publication
(10) Pub. No.: US 2023/0201506 A1
(43) Pub. Date: Jun. 29, 2023

(54) RESPIRATORY CANNULA SYSTEMS AND METHODS OF USE

(71) Applicant: The Board of Supervisors of Louisiana State University and Agricultural and Mechanical College, Baton Rouge, LA (US)

(72) Inventors: Rohan R. Walvekar, Metairie, LA (US); Michael Dunham, New Orleans, LA (US); Tyler Crosby, Bush, LA (US)

(21) Appl. No.: 17/926,906

(22) PCT Filed: May 20, 2021
(86) PCT No.: PCT/US2021/033428

§ 371 Date: Nov. 21, 2022

Related U.S. Application Data
(60) Provisional application No. 63/028,107, filed on May 21, 2020.

(51) Int. Cl. A61M 16/04; A61M 16/16

(52) U.S. Cl. CPC A61M 16/0427; A61M 16/0465; A61M 16/16

(57) ABSTRACT

The present invention is directed to an improved cannula systems for use with tracheostomies and endotracheal tubes. The presently disclosed cannula system permits efficient delivery of an external substance or compound into the respiratory system of a patient. A method of use of the cannula system is also disclosed herein.

### CROSS-REFERENCE TO RELATED APPLICATIONS

This application claims priority from U.S. Provisional Application No. 63/028,107 filed on May 21, 2020, the entire contents of which are incorporated herein by reference.

All patents, patent applications, and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

Not applicable.

### FIELD OF THE INVENTION

The present invention is directed to design and functional improvements of the cannula portion of tracheostomy and endotracheal tubes.

### BACKGROUND OF THE INVENTION

Tracheostomy cannulas and endotracheal tubes are commonly used devices for creating or securing a temporary or permanent breathing passage. However current tracheostomy and endotracheal systems exhibit significant inefficiencies including problems with humidification of incoming air, delivery of oxygen, and delivery of pharmaceutical agent.

### SUMMARY OF THE INVENTION

Aspects of the invention are drawn to a respiratory cannula system.

For example, embodiments can comprise an outer cannula and an inner cannula. In embodiments, the outer cannula comprises an outer cannula body, an outer cannula lumen, and a port in the form of a hole, gap, notch, or channel extending through the outer cannula body. In embodiments, the inner cannula further comprises an inner cannula body and an inner cannula lumen. In embodiments, the outer cannula is configured to receive and hold at least a portion of the inner cannula body within at least a portion of the outer cannula lumen; and a chamber exists in the outer cannula lumen when the inner cannula is disposed therein, the chamber being in fluid communication with a patient's respiratory system.

In embodiments, the outer cannula lumen comprises a diameter that is slightly larger than a diameter of the inner cannula body; and the chamber exists as a space within the outer cannula lumen when at least a portion of the inner cannula body resides therein.

In embodiments, the diameter of the inner cannula body is about 80% of the diameter of the outer cannula lumen.

In embodiments, the outer cannula lumen comprises a diameter of about 10 mm or less. For example, the outer cannula lumen comprises a diameter of about 6 mm, about 7 mm, about 8 mm, about 9 mm, or about 10 mm.

In embodiments, the inner cannula body comprises a diameter of about 8.5 mm or less. For example, the inner cannula body comprises a diameter of about 4.5 mm, about 5.5 mm, about 6.5 mm, about 7.5 mm, or about 8.5 mm.

In embodiments, the outer cannula lumen comprises a diameter of about 10 mm or less and the inner cannula body comprise a diameter of about 8.5 mm or less. For example, the outer cannula lumen comprises a diameter of about 8 mm and inner canula body comprises a diameter of about 6.5 mm.

In embodiments, the port comprises a tubular extension extending from an external surface of the outer cannula body.

In embodiments, the port permits fluid communication between an external source and the chamber.

In embodiments, the port comprises a valve to selectively control fluid communication with the chamber.

In embodiments, the port further comprises a Luer-lock or Luer-taper hub.

In embodiments, the outer cannula comprises at least two ports.

In embodiments, the inner cannula body further comprises at least one vent that is configured to permit fluid communication between the inner cannula lumen and the chamber, the port, or a combination thereof. For example, the at least one vent comprises a plurality of perforations disposed along the inner cannula body.

In embodiments, the external source is configured to introduce oxygen, humidified air, a pharmaceutical agent, or a combination thereof into the chamber. For example, the pharmaceutical agent comprises one or more aerosolized drugs. For example, the pharmaceutical agent comprises albuterol, dexamethasone, tobramycin, any other medication that can be delivered via a nebulizer, or a combination thereof.

In embodiments, the outer cannula, inner cannula, or both comprise a medical grade metal, a medical-grade polymer, or a combination thereof. For example, the medical grade metal comprises stainless steel, titanium, tantalum, gold, platinum, palladium, or a combination thereof. For example, the outer cannula, inner cannula, or both comprise a silicone elastomer, sterilizable plastic, polytetrafluoroethylene, polyether block amide, polyvinyl chloride, or a combination thereof.

In embodiments, the outer cannula, inner cannula, or both are reusable.

In embodiments, the outer cannula, inner cannula, or both are disposable.

Aspects of the invention are also drawn towards a method of delivering humidified air to a patient with a tracheostomy. For example, the method comprises obtaining a respiratory cannula system as described herein, inserting the outer cannula through a stoma and into the trachea of a patient; inserting the inner cannula into the outer cannula lumen; attaching an external source of humidified air to the port of the outer cannula; starting the flow of humidified air through the port, into the chamber, and into the respiratory system of the patient.

Aspects of the invention are still further drawn towards a method of delivering oxygen to a patient with a tracheostomy. For example, the method comprises obtaining a respiratory cannula system as described herein; inserting the outer cannula through a stoma and into the trachea of a patient; inserting the inner cannula into the outer cannula lumen; attaching an external source of oxygen to the port of the outer cannula; starting the flow of oxygen through the port, into the chamber, and into the respiratory system of the patient.

Still further, aspects of the invention are drawn towards a method of delivering a pharmaceutical agent to a patient with a tracheostomy. For example, the method comprises obtaining a respiratory cannula system as described herein; inserting the outer cannula through a stoma and into the trachea of a patient; inserting the inner cannula into the outer cannula lumen; attaching an external source of the pharmaceutical agent to the port of the outer cannula; starting the flow of the pharmaceutical agent through the port, into the chamber, and into the respiratory system of the patient.

Aspects of the invention are also drawn towards an outer cannula for use in a respiratory cannula system. For example, embodiments comprise an outer cannula body, an outer cannula lumen, and a port in the form of a hole, gap, notch, or channel extending through the outer cannula body; the outer cannula being configured to receive and hold at least a portion of an inner cannula body within at least a portion of the outer cannula lumen; the outer cannula lumen comprising a diameter that is slightly larger than a diameter of the inner cannula body such that a chamber exists within the outer cannula lumen when an inner cannula body resides therein; the port being in fluid communication with the chamber; and the chamber being in fluid communication with a patient's respiratory system.

In embodiments, the port comprises a tubular extension extending from an external surface of the outer cannula body.

Aspects of the invention are also drawn towards an inner cannula for use in a respiratory cannula system. For example, embodiments comprise an inner cannula body and an inner cannula lumen; at least a portion of the inner cannula body being configured to reside within an outer cannula lumen comprising a diameter that is slightly larger than a diameter of the inner cannula body such that a chamber exists within the outer cannula lumen when at least a portion of the inner cannula body is disposed therein.

In embodiments, the inner cannula body further comprises at least one vent that is configured to permit fluid communication between the inner cannula lumen and the chamber.

In embodiments, the at least one vent comprises a plurality of perforations disposed along the inner cannula body.

Further, aspects of the invention are drawn towards a kit for use in a respiratory cannula system. For example, embodiments comprise an outer cannula further comprising (1) an outer cannula body, (2) an outer cannula lumen, and (3) a port in the form of a hole, gap, notch, or channel extending through a portion of the outer cannula body; an inner cannula further comprising an inner cannula body and an inner cannula lumen; wherein the outer cannula is configured to receive and hold at least a portion of the inner cannula body within at least a portion of the outer cannula lumen; and a chamber exists in the outer cannula lumen when the inner cannula is disposed therein, the chamber being in fluid communication with a patient's respiratory system. Embodiments can comprise instructions for use of the kit. Embodiments can further comprise a pharmacological agent for distribution into the respiratory system of the patient through the port of the outer cannula.

Other objects and advantages of this invention will become readily apparent from the ensuing description.

### BRIEF DESCRIPTION OF THE FIGURES

Certain illustrations, charts, or flow charts are provided to allow for a better understanding for the present invention. It is to be noted, however, that the drawings illustrate only selected embodiments of the inventions and are therefore not to be considered limiting of scope. Additional and equally effective embodiments and applications of the present invention exist.

FIG. 1 shows a traditional tracheostomy system modified with an oxygen adaptor for delivery of oxygen.

FIG. 2A shows a traditional tracheostomy system retrofitted with a mask adapter.

FIG. 2B shows the traditional tracheostomy system and mask adapter of FIG. 2A, wherein the mask adapter further includes tubing.

FIG. 2C shows the traditional tracheostomy system and mask adapter of FIG. 2A, with a nebulizer attached to the mask adapter.

FIG. 3A provides a front perspective view of a tracheostomy cannula system in accordance with one embodiment of the present invention.

FIG. 3B shows the tracheostomy cannula system of FIG. 3A with the outer cannula shown in phantom to reveal the body of the inner cannula residing therein.

FIG. 4 shows a front perspective view of the inner cannula used in the cannula system of FIG. 3A.

FIG. 5 shows a front perspective view of the outer cannula used in the cannula system of FIG. 3A.

FIG. 6A provides a schematic side perspective view of an cannula system under one embodiment of the present invention.

FIG. 6B shows a bottom view the FIG. 6A schematic.

FIG. 6C shows a transverse cross-sectional view taken through plane B-B of the FIG. 6B schematic.

FIG. 6D shows a longitudinal cross-sectional view taken through plane A-A of the FIG. 6B schematic.

FIG. 7A provides a schematic side perspective view of an outer cannula under one exemplary embodiment.

FIG. 7B shows a side view of the FIG. 7A schematic.

FIG. 7C provides a front view of the FIG. 7A schematic. Non-limiting, exemplary dimensions are shown in mm.

FIG. 7D shows a top view of the FIG. 7A schematic. Non-limiting, exemplary dimensions are shown in mm.

FIG. 8A provides a schematic front perspective view of an inner cannula under one embodiment.

FIG. 8B shows a side view of the FIG. 8A schematic. Non-limiting, exemplary dimensions are shown in mm.

FIG. 8C shows a front view of the FIG. 8A schematic.

FIG. 8D provides a top view of the FIG. 8A schematic. Non-limiting, exemplary dimensions are shown in mm.

FIG. 9 provides a side perspective view of the top portion of an inner cannula under one embodiment. A port or vent can be seen extending through the body of the inner cannula.

FIGS. 10A and 10B provide computational fluid dynamics (CFD) of an exemplary embodiment of the presently disclosed cannula system.

### DETAILED DESCRIPTION OF THE INVENTION

### Abbreviations and Definitions

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a,” “an,” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The terms “comprising” and “including” and “having” and “involving” (and similarly “comprises,” “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

For purposes of the present disclosure, it is noted that spatially relative terms, such as “up,” “down,” “right,” “left,” “beneath,” “below,” “lower,” “above,” “upper” and the like, can be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device can be otherwise oriented (rotated 90 degrees or at other orientations) and the spatially relative descriptors used herein interpreted accordingly.

The terms “subject” and “patient” as used herein include all members of the animal kingdom including, but not limited to, mammals, animals (e.g., cats, dogs, horses, swine, etc.) and humans.

The term “caretaker” as used herein refers to any person, group, or entity who has assumed responsibility to care for the subject or patient or to prepare the therapeutic device for use. By way of non-limiting example, a caretaker can include a physician, a nurse, a clinician, a pharmacist, a physician's assistant, any employee of a clinical facility, a family member of the subject, a friend or acquaintance of the subject, an employee of the subject, or any other person, group, or entity who assumes responsibility to care for the subject. In certain instances, the subject can act as a caretaker, such as when tending to his or her own wounds.

### Description of Selected Embodiments

Disclosed herein is an improved cannula system for tracheostomies and endotracheal tubes. In various exemplary embodiments, the present invention provides significant improvements including oxygen and drug delivery capabilities. The disclosed embodiments further improve the utility, efficiency and capabilities of these indwelling cannulas while also reducing potential complications including mucous plugging.

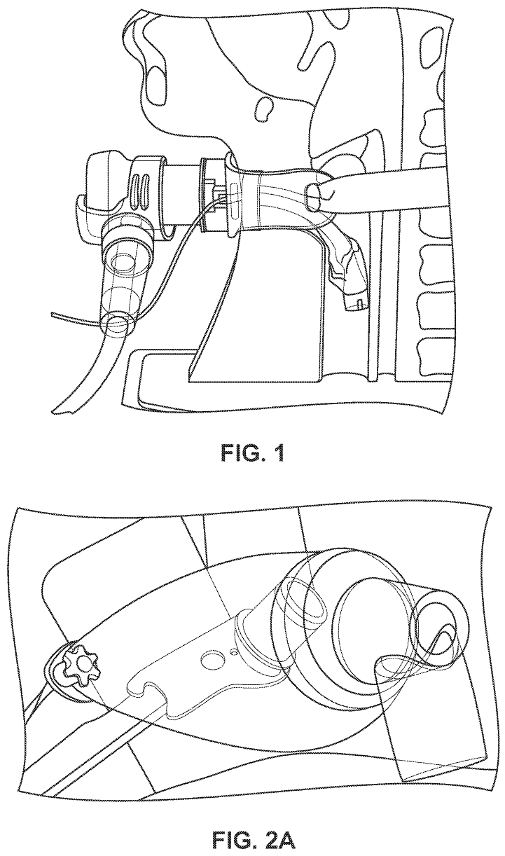

FIG. 1 shows a traditional tracheostomy system modified with an off-the-shelf oxygen adaptor for delivery of oxygen. As can be seen, the oxygen adapter adds substantial size and bulk to the tracheostomy, which can be uncomfortable for the patient. Further, the oxygen adapter requires heavy tubing attached thereto, which can create additional drag on the tracheostomy tube, increasing the risk of cannula dislodgement. Such a configuration can also increase the potential for damage to the existing stoma or create additional wounds such as through the burying of the tracheostomy into the skin of the patient.

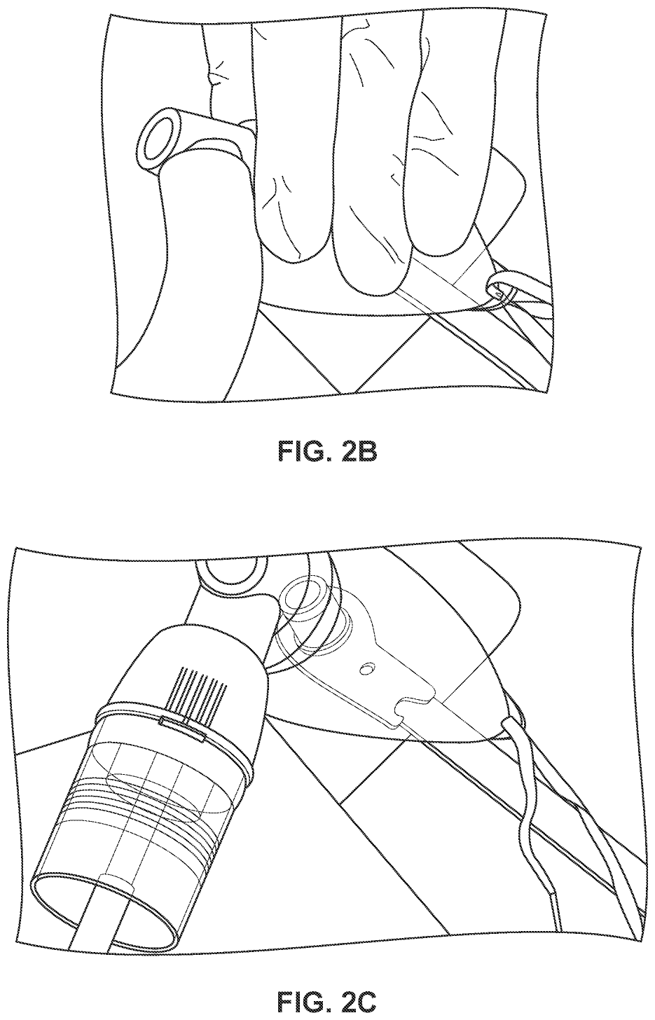

FIG. 2A shows another traditional tracheostomy system that has been retrofitted with a mask adapter. Once again, the mask introduces a large addition to the standard tracheostomy system, which can be uncomfortable, particularly when the patient wears the mask for extended periods of time. In addition, the mask can become misaligned easily, requiring constant adjustment to ensure adequate delivery to the patient. As further shown in FIGS. 2B and 2C, when the mask is equipped with oxygen lines or a nebulizer, respectively, the disadvantages are exacerbated due to the additional size and weight of the accessories. As best seen in FIG. 2A, gaps can easily form between the neck of the patient and the mask adapter, resulting in inefficient delivery of oxygen, humidified air, or any pharmaceutical compounds. The lack of an effective seal can also make it difficult for a caretaker to adequately determine the dosage of a particular compound or substance that is actually received by the patient. In addition, masks such as that pictured in FIGS. 2A-2C are designed to be tied around the neck of a patient, which can be problematic when patients have conditions or additional medical equipment (e.g. patients with head neck flaps). In such patients, the caretaker must find an alternate method of securing the mask to the patient, which often includes anchoring the mask to the chest or neck using medical tape. This alternate means of attachment is typically less efficient, resulting in further exacerbation of the disadvantages mentioned above.

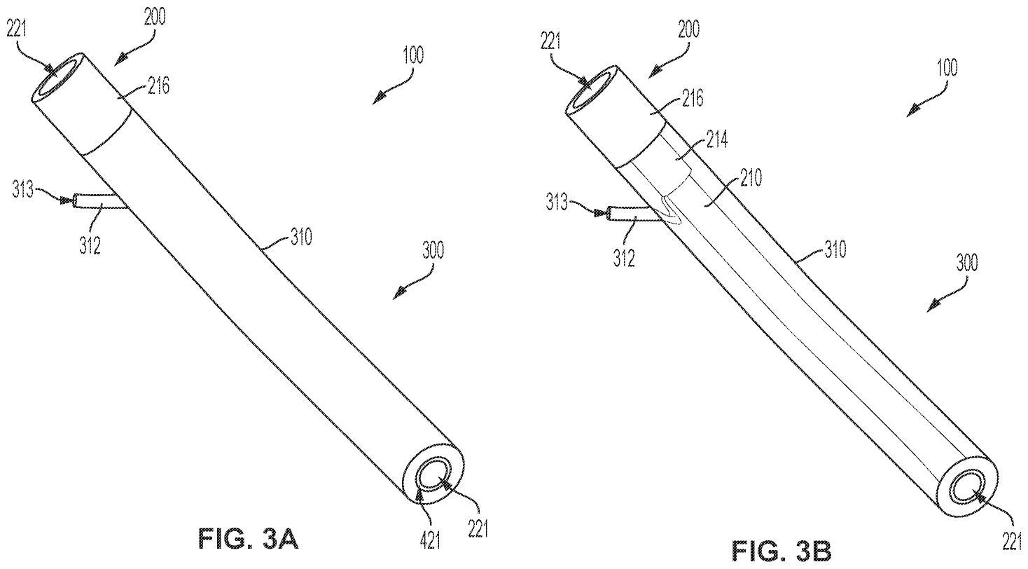

FIG. 3A provides a front perspective view of a tracheostomy cannula system 100 in accordance with one embodiment of the present invention. FIG. 3B provides the same view as FIG. 3A, but with the outer cannula 300 shown in phantom to reveal the length of the inner cannula 200 residing therein. The head 216 of the inner cannula 200 can be seen resting on the top most portion of the outer cannula 300. The inner cannula 200 comprises a lumen 221 that begins at the top of the head 216 of the inner cannula 200 extends along the length of the inner cannula to exit via the terminal portion of the body 210. The outer cannula 300 comprises at least one passthrough, hole, or channel, which forms a port 313 that extends through the wall 350 of the outer cannula body 310. In the pictured embodiments, the port 313 comprises a tubular extension 312 that extends upward at an angle from the wall 350 of the outer cannula body 310.

The outer cannula further comprises a lumen (more clearly seen at 321 of FIG. 5), which is configured to receive and hold at least a portion of the inner cannula body 210. As shown, the outer cannula lumen 321 comprises a diameter that is larger than that of at least a portion of the inner cannula body 210. The diameter of the outer cannula can be sufficiently large such that a chamber 421 is formed between the exterior surface of the inner cannula body 210 and the interior surface of the outer cannula body 310 when at least a portion of the internal cannula body 210 is disposed within the lumen 321 of the outer cannula 300.

In embodiments, the port 313 is in fluid communication with the lumen 321 of the outer cannula 300 such that the port 313 is in fluid communication with the chamber 421 when the body of the inner cannula 210 is disposed within the lumen of the outer cannula 321.

In embodiments, the tubular extension 312 of the port 313 extends at about a 45° angle from the wall 350 of the outer cannula 300. The tubular extension 212 can extend at any angle that provides convenient attachment/detachment of an external source thereto. The tubular extension 312 can be substantially perpendicular to the wall 350 of the outer cannula 300. In certain embodiments, the tubular extension 312 extends from the wall 350 at an angle of between about 15° to about 90°, inclusive. In embodiments, the tubular extension 312 extends at an angle of between about 30° and about 70°, inclusive. The tubular extension 312 can extend at an angle of between about 40° to about 60°, inclusive. In embodiments, the tubular extension 312 extends from the wall 350 at an angle that ranges from about 45° to about 55°. The tubular extension 312 can extend at an angle of about 20°, about 30°, about 40°, about 50°, about 60°, about 70°, about 80°, or about 90°.

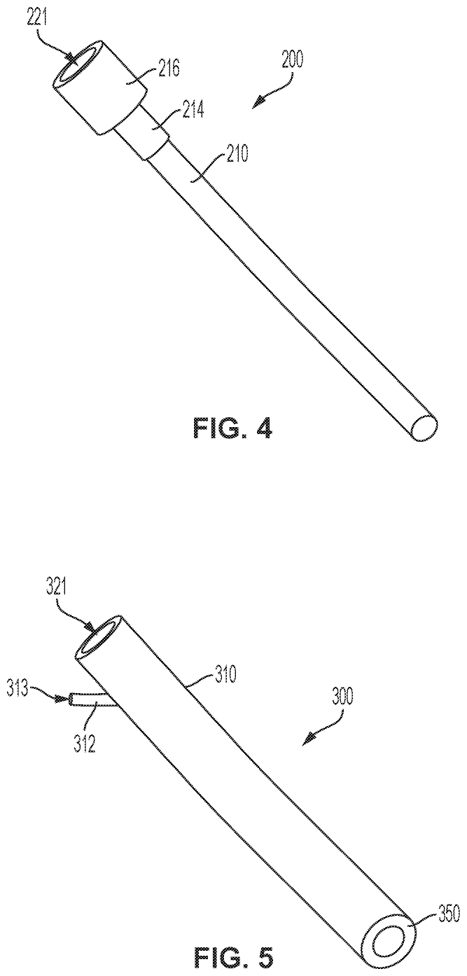

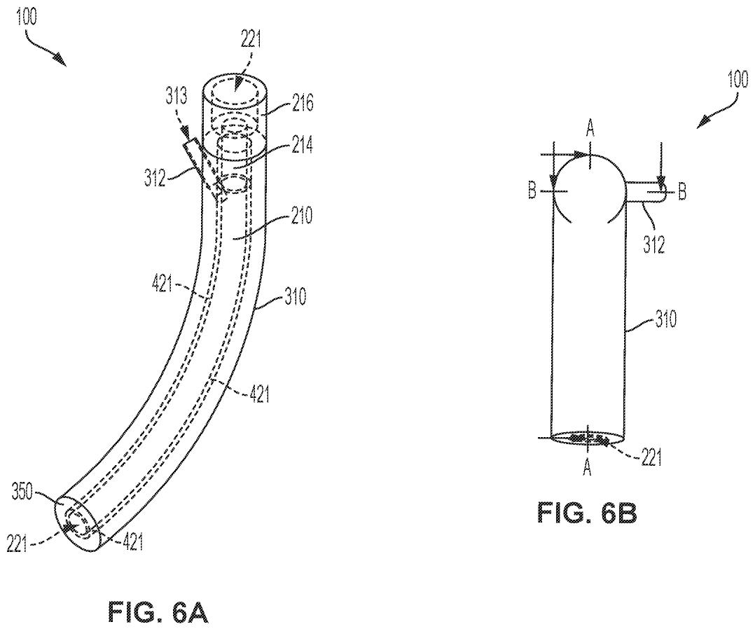

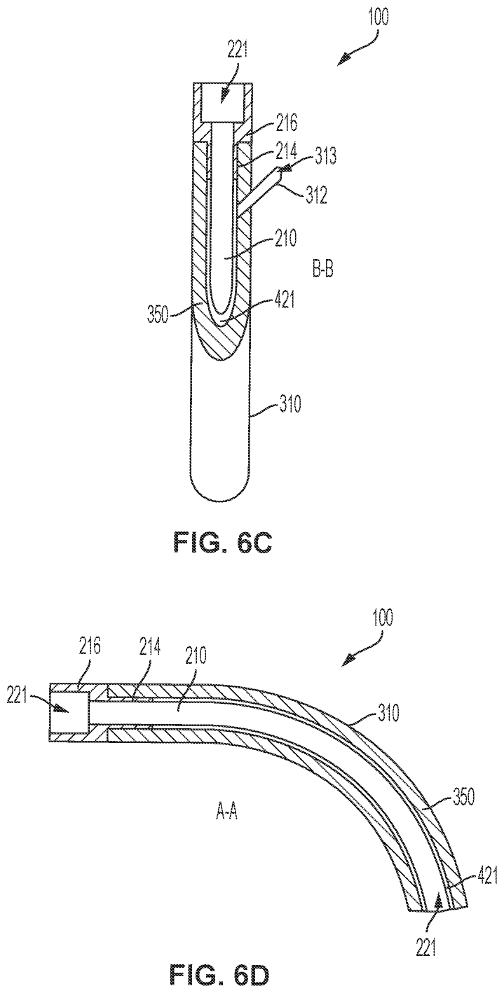

FIG. 4 shows a front perspective view of the inner cannula 200 used in the cannula system 100 of FIG. 3A. As can be seen, the inner cannula 200 can comprise a substantially circular cross sectional shape with an exterior wall surface and an interior wall surface such that a lumen 221 extends therethrough. The exemplary embodiment comprises a head 216 at the top most portion of the internal cannula 200. In the pictured embodiment, a neck 214 can be seen immediately below the head 216 of the internal cannula 200. A body 210 can also bee seen extending downward from the neck 214. Alternate embodiments are envisioned that do not comprise a head 216 or neck 214. Certain embodiments lack both the head 216 and the neck 214. In the pictured embodiment, the head 216 is wider than the neck 214, and the neck 214 is wider than the body 210. In embodiments, the head 216 is configured to reside outside of the lumen of the outer cannula 321. The neck 214, body 210, or both can be configured to reside within the lumen of the outer cannula 321. In embodiments, the diameter of the neck 214 is configured to prevent the formation of a space between the interior wall of the outer cannula 300 and the exterior wall of the neck 214. The neck 214 can be configured to extend into the lumen of the outer cannula 321 to just above the port 313 of the outer cannula 300. When such an inner cannula embodiment resides within the lumen of the outer cannula 321, the neck 214 prevents the chamber (more clearly seen at 421 of FIG. 6A) from being in fluid communication with the air surrounding the top surface of the cannula system 100. As such, any substance introduced through the port 313 is forced to travel down the chamber 421 and exit the bottom portion of the cannula system 100.

FIG. 5 shows a front perspective view of the outer cannula 300 used in the cannula system of FIG. 3A. As can be seen, the outer cannula 300 comprises an outer cannula wall 350. The outer cannula 300 can comprise a substantially circular cross sectional shape with an exterior wall surface and an interior wall surface such that a lumen 321 extends therethrough. As shown, the outer cannula can further comprises a tubular extension 312 with a passthrough, hole, or channel that extends through the wall 350 of the outer cannula to form a port 313 that permits fluid communication between an external source and the lumen of the outer cannula 321.

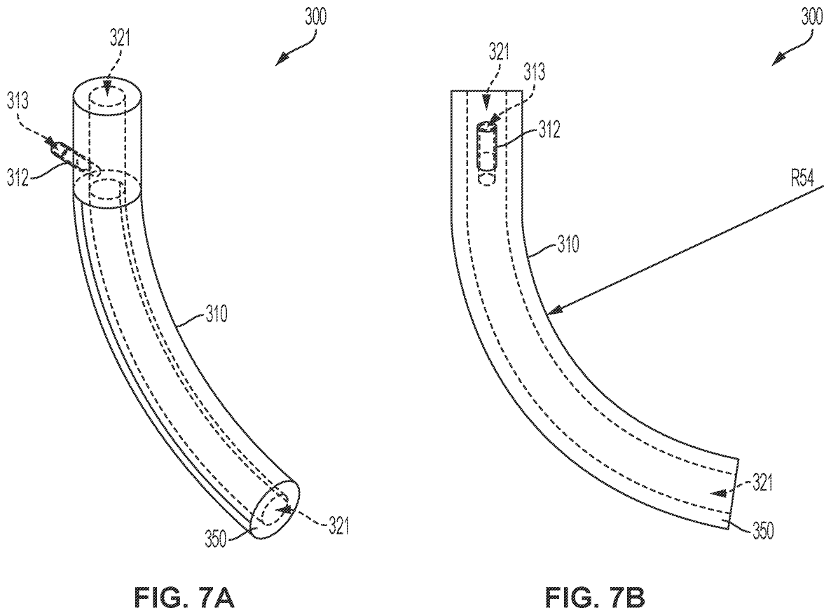

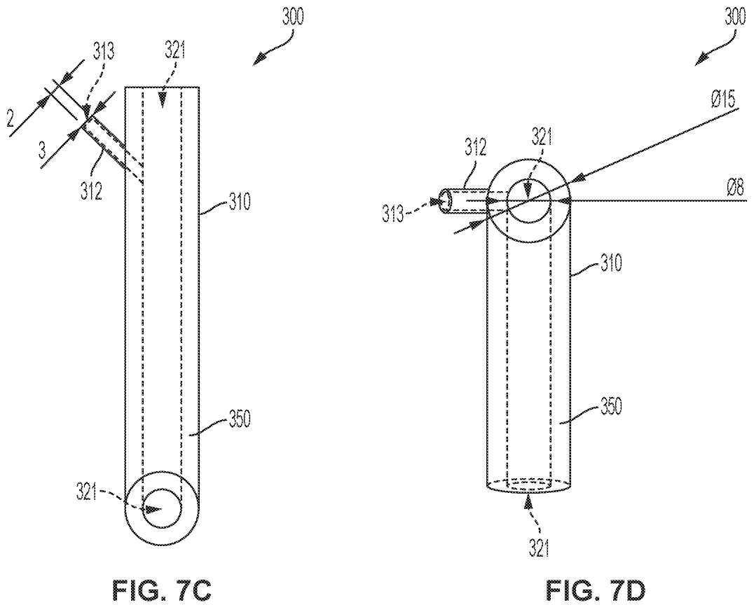

FIG. 6A provides a schematic side perspective view of an cannula system 100 under one embodiment of the present invention. An inner cannula 200 can be seen residing with the lumen (more clearly seen at 321 of FIGS. 5 and 7A-7D) of the outer cannula 300. The walls of the inner 200 and outer 300 cannulas are shown in phantom to reveal the lumen of the inner cannula 221 and the chamber 421 formed between the exterior walls of the inner cannula body 210 and the interior wall 350 of the outer cannula body 310. This embodiment reveals the manner in which the port 313 is in fluid communication with the chamber 421 when the inner cannula body 210 is disposed within the lumen of the outer cannula 321.

FIG. 6B shows a bottom view the FIG. 6A schematic. The tubular extension 312 is shown extending laterally from the body 310 of the outer cannula.

FIG. 6C shows a transverse cross-sectional view taken through plane B-B of the FIG. 6B schematic, and FIG. 6D shows a longitudinal cross-sectional view taken through plane A-A of the FIG. 6B schematic. The head 216 of the inner cannula is shown resting on the top-most portion of the outer cannula body 310. The neck 214 of the inner cannula can be seen extending partially down into the lumen of the outer canula to an area above the location where the port 313 breaches the wall 350 of the outer cannula (visible in FIG. 6C). The chamber 421 between the exterior wall of the inner cannula body 210 and the interior wall 350 of the outer cannula is clearly seen in the FIGS. 6C and 6D schematics. As can be seen, the chamber 421 surrounds the exterior wall of the inner cannula body 210 and is in fluid communication with the terminal point of the cannula system 100.

FIG. 7A provides a schematic side perspective view of an outer cannula 300 under one exemplary embodiment. The wall 350 is shown in phantom to reveal the lumen 321 of the outer cannula extending therethrough, and the port 313 can be seen extending through the wall 350 and into the lumen 321 of the outer cannula 300.

FIG. 7B shows a side view of the FIG. 7A schematic. The body 310 of the outer cannula 300 is shown with an exemplary radius of curvature of about 54°. In certain embodiments, the radius of curvature is about the same as that of traditional, off-the-shelf cannulas. The radius of curvature can range from about 45° to about 65°. In embodiments, the radius of curvature of the outer cannula body is about 50°, about 51°, about 52°, about 53°, about 54°, about 55°, about 56°, about 57°, about 58°, about 59°, or about 60°.

FIG. 7C provides a front view of the FIG. 7A schematic. In this particular embodiment, the tubular extension 312 comprises a diameter of about 3 mm and the port 313 comprises a diameter of about 2 mm. In embodiments, the diameter of the tubular extension 312 can comprise any diameter that is sufficient for connection to an external source. The diameter of the tubular extension 312 can range from about 0.5 mm up to about 10 mm. In embodiments, the tubular extension 312 comprises a diameter in the range of about 2 mm to about 6 mm. The diameter of the tubular extension 312 can be about 1 mm, about 2 mm, about 3 mm, about 4 mm, about 6 mm, about 7 mm, about 8 mm, about 9 mm, or about 10 mm. In embodiments, the diameter of the port 313 can be any diameter that permits unobstructed flow of air therethrough. The diameter of the port 313 can be less than 1 mm. In embodiments, the diameter of the port 313 can be as large as 5 mm. The port 313 can comprise a diameter of between about 0.25 mm up to about 4 mm. In certain embodiments, the port 313 comprises a diameter of about 0.25 mm, about 0.5 mm, about 1.0 mm, about 1.5 mm, or about 2 mm.

FIG. 7D shows a top view of the FIG. 7A schematic. In this particular embodiment, the diameter of the outer cannula 300 is about 15 mm, and the diameter of the lumen 321 is about 8 mm. In embodiments, the lumen 321 comprises any diameter that permits the outer cannula 300 to receive and hold an inner cannula. The diameter of the lumen 321 can range from about 2 mm up to about 16 mm. In embodiments, the diameter of the lumen 321 is less than 2 mm. The lumen 321 can comprise a diameter that is larger than 16 mm. In certain embodiments, the diameter of the lumen is about 3 mm, about 4 mm, about 5 mm, about 6 mm, about 7 mm, about 8 mm, about 9 mm, about 10 mm, about 11 mm, about 12 mm, about 13 mm, about 14 mm, or about 15 mm. The outer cannula 300 can comprise any diameter that is suitable for use in a subject. In embodiments, the outer cannula 300 comprises a diameter that is up to about 30 mm. The diameter of the outer cannula 300 can be as small as about 5 mm. In certain embodiments, the diameter of the outer cannula 300 ranges from about 10 mm to about 20 mm. The outer cannula 300 can comprise a diameter of about 10 mm, about 11 mm, about 12 mm, about 13 mm, about 14 mm, about 15 mm, about 16 mm, about 17 mm, about 18 mm, about 19 mm, or about 20 mm.

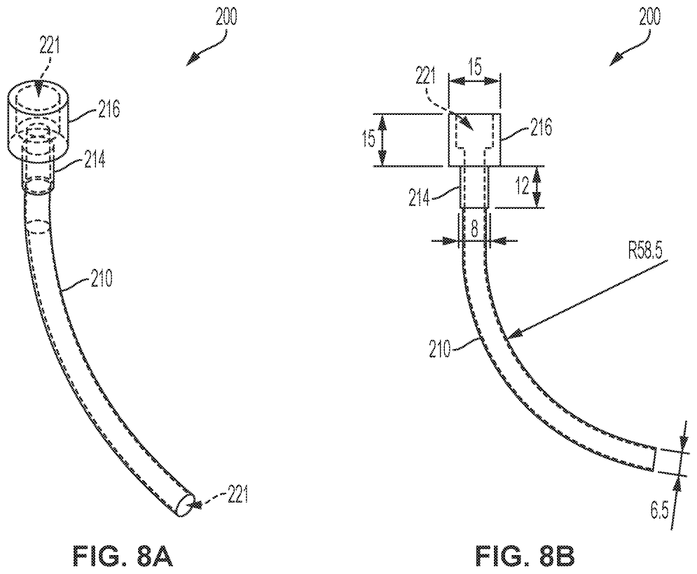

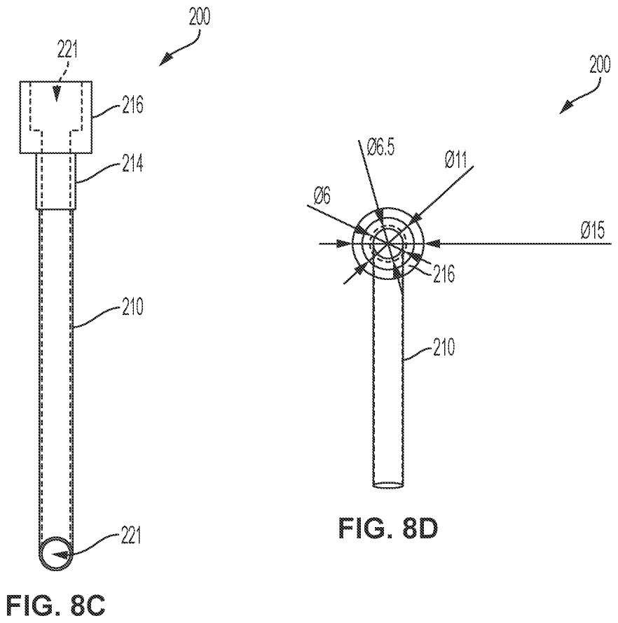

FIG. 8A provides a schematic front perspective view of an inner cannula 200 under one embodiment, and FIG. 8B shows a side view of the FIG. 8A schematic. Similarly, FIG. 8C shows a front view of the FIG. 8A schematic. The head 216, neck 214, and body 210 of the inner cannula 200 are all shown with their walls in phantom to reveal the lumen 221 of the internal cannula. As can be seen, the lumen 221 of the internal cannula 221 can comprise different diameters. For instance, the topmost portion of the head 216 is shown with relatively thin walls as compared to the lower portion of the head 216 such that the lumen 221 is wider at the top portion than the lower portion of the head 216.

In one particular embodiment, the head 216 comprise a length of about 15 mm and a width of about 15 mm. In alternate embodiments, the head 216 comprise a length of less than about 5 mm. The head 216 can comprise a length of greater than 15 mm. In embodiments, the length of the head 216 ranges from about 5 mm to about 25 mm. The head 216 can comprise a length of between about 10 mm to about 20 mm. In embodiments, the head 216 comprises a length of about 10 mm, about 11 mm, about 12 mm, about 13 mm, about 14 mm, about 16 mm, about 17 mm, about 18 mm, about 19 mm, or about 20 mm.

In one embodiment, the head 216 comprise a cross-sectional diameter of about 15 mm. In alternate embodiments, the head 216 comprise a diameter of less than about 5 mm. The head 216 can comprise a diameter of greater than 15 mm. In embodiments, the diameter of the head 216 ranges from about 5 mm to about 25 mm. The head 216 can comprise a diameter of between about 10 mm to about 20 mm. In embodiments, the head 216 comprises a diameter of about 10 mm, about 11 mm, about 12 mm, about 13 mm, about 14 mm, about 16 mm, about 17 mm, about 18 mm, about 19 mm, or about 20 mm.

In one particular embodiment, the neck 214 comprise a length of about 12 mm and a width of about 8 mm. In alternate embodiments, the neck 214 comprise a length of less than about 1 mm. The neck 214 can comprise a length of greater than 12 mm. In embodiments, the length of the neck 214 ranges from about 1 mm to about 25 mm. The neck 214 can comprise a length of between about 7 mm to about 17 mm. In embodiments, the neck 214 comprises a length of about 5 mm, about 6 mm, about 7 mm, about 8 mm, about 9 mm, about 10 mm, about 11 mm, about 12 mm, about 13 mm, about 14 mm, about 16 mm, about 17 mm, about 18 mm, about 19 mm, or about 20 mm.

In one embodiment, the neck 214 comprise a cross-sectional diameter of about 8 mm. In alternate embodiments, the neck 214 comprise a diameter of less than about 1 mm. The neck 214 can comprise a diameter of greater than 8 mm. In embodiments, the diameter of the neck 214 ranges from about 1 mm to about 20 mm. The neck 214 can comprise a diameter of between about 2 mm to about 16 mm. In embodiments, the neck 214 comprises a diameter of about 1 mm, about 2 mm, about 3 mm, about 4 mm, about 6 mm, about 7 mm, about 8mm, about 9 mm, about 10 mm, about 11 mm, about 12 mm, about 13 mm, about 14 mm, about 16 mm, about 17 mm, about 18 mm, about 19 mm, or about 20 mm.

In one embodiment, the cross sectional diameter of the body 210 of the internal cannula is about 6.5 mm. In alternate embodiments, the body 210 comprise a diameter of less than about 1 mm. The body 210 can comprise a diameter of greater than 6.5 mm. In embodiments, the diameter of the body 210 ranges from about 1 mm to about 15 mm. The body 210 can comprise a diameter of between about 1 mm to about 12 mm. In embodiments, the body 210 comprises a diameter of about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm.

The body 210 of the inner cannula 200 is shown with an exemplary radius of curvature of about 58.5°. In certain embodiments, the radius of curvature is about the same as that of traditional, off-the-shelf cannulas. The radius of curvature can range from about 45° to about 75°. In embodiments, the radius of curvature of the outer cannula body is about 50°, about 51°, about 52°, about 53°, about 54°, about 55°, about 56°, about 57°, about 58°, about 59°, about 60°, about 61°, about 62°, about 63°, about 64°, or about 65°.

FIG. 8D provides a top view of the FIG. 8A schematic. Non-limiting, exemplary dimensions of the various diameters are shown in mm. In one embodiment, the portion of the interior cannula lumen 221 within the head 216 comprises at least two different diameters. The lumen 221 within the topmost portion of the head 216 can comprise a diameter of about 11 mm. In embodiments, the lumen 221 within the topmost portion of the head 216 comprises a diameter of greater than 11 mm. The diameter of the lumen 221 within the topmost portion of the head 216 can be les than 11 mm. In certain embodiments, the diameter of the lumen 221 within the topmost portion of the head 216 ranges from between about 2 mm to about 25 mm. The diameter of the lumen 221 within the topmost portion of the head 216 can be between about 5 mm and about 15 mm. In embodiments, the lumen 221 within the topmost portion of the head 216 comprises a diameter of about 5 mm, about 6 mm, about 7 mm, about 8 mm, about 9 mm, about 10 mm, about 11 mm, about 12 mm, about 13 mm, about 14 mm, or about 15 mm.

The lumen 221 within the lowest portion of the head 216 can comprise a diameter of about 6 mm. In embodiments, the lumen 221 within the lowest portion of the head 216 comprises a diameter of greater than 6 mm. The diameter of the lumen 221 within the lowest portion of the head 216 can be less than 6 mm. In certain embodiments, the diameter of the lumen 221 within the lowest portion of the head 216 ranges from between about 0.5 mm to about 20 mm. The diameter of the lumen 221 within the lowest portion of the head 216 can be between about 1 mm and about 15 mm. In embodiments, the lumen 221 within the lowest portion of the head 216 comprises a diameter of about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, or about 15 mm. The lumen 221 within the lowest portion of the head 216 can comprises a diameter that is equal to that the diameter of the lumen 221 within the body 210 of the internal cannula.

In an embodiment, the diameter of the lumen 221 within the body 210 of the internal cannula is about 6 mm. In embodiments, the lumen 221 within the body 210 comprises a diameter of greater than 6 mm. The diameter of the lumen 221 within body 210 can be less than 6 mm. In certain embodiments, the diameter of the lumen 221 within the body 210 ranges from between about 0.5 mm to about 20 mm. The diameter of the lumen 221 within the body 210 can be between about 1 mm and about 15 mm. In embodiments, the lumen 221 within the body 210 comprises a diameter of about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, or about 15 mm.

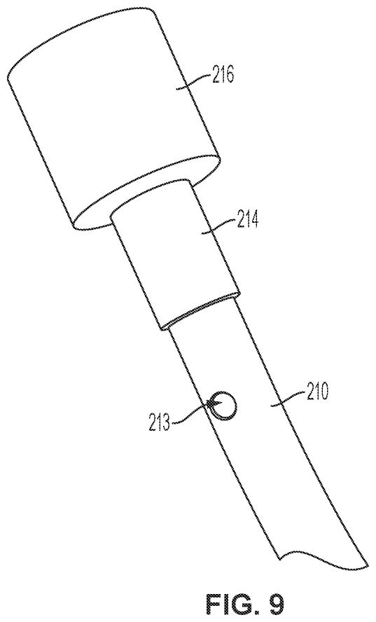

FIG. 9 provides a side perspective view of the top portion of an inner cannula 200 under one embodiment. A port or vent 213 can be seen extending through the body of the inner cannula. In embodiments, the port 213 of the internal cannula can be aligned with the port 313 of the outer cannula 300 when the body of the internal cannula 210 resides within the lumen 321 of the outer cannula 300.

As shown in the various exemplary embodiments disclosed herein, the cannula system 100 permits simple and effective delivery of an external substance or compound into the respiratory system of a patient in need thereof The presently disclosed cannula system 100 allows for such delivery without the need for bulky, cumbersome, or uncomfortable adapters or attachments that are presently available on the market (see, for example FIGS. 1, 2A, 2B, and 2C).

In use, the cannula system 100 can be implanted within a patient's trachea such that the lumen 221 of the internal cannula 200 and the chamber 421 are in fluid communication with the patient's respiratory system. In addition, the port 313 of the outer cannula 300 permits fluid communication between an external source and the chamber 421. As such, the port 313 permits the introduction of a substance or compound into the respiratory system of a patient while obviating the need to remove the inner cannula or otherwise interfere with the flow within the inner cannula.

In operation, the tubular extension 312 can be connected to an external source such that the external source delivers a substance or compound through the port 313, into the chamber 421, and ultimately into the respiratory system of the patient.

In various exemplary embodiments, the external source can be configured to introduce any substance or compound that can be useful in treating a patient. By way of non-limiting example, the external source can be configured to introduce oxygen, humidified air, a pharmaceutical agent, or a combination thereof into the chamber 421. Exemplary pharmaceutical agents include, but are not limited to albuterol, dexamethasone, tobramycin, any other medication that can be delivered via a nebulizer, or a combination thereof

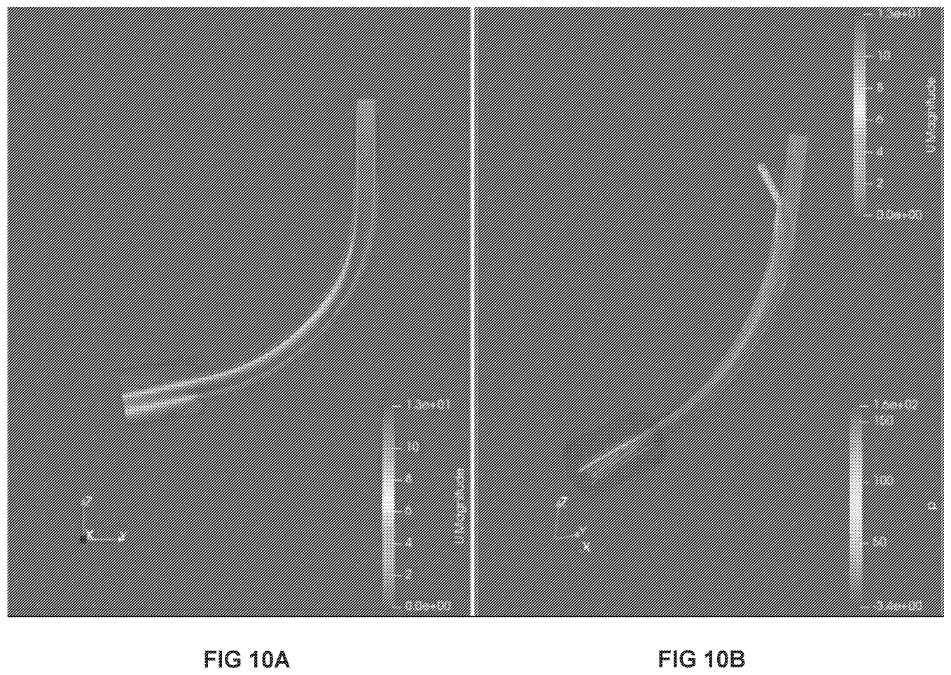

FIGS. 10A and 10B provide computational fluid dynamics (CFD) of an exemplary embodiment of the presently disclosed cannula system. The CFD demonstrates delivery or flow into the distal airway at physiologic volumetric flows. As shown in these figures, the presently disclosed cannula system 100 appears to permit mixing of the inner and outer flows beyond the end of the tracheostomy tube. Further, without wishing to be bound by theory, laminar flow in the outer chamber moving around the inner cannula indicates that mixing flows in the proximal tube can facilitate clearing of secretions, thereby preventing the buildup of mucus within the cannula system 100.

In certain embodiments, the inner cannula can be provided with one or more vents, wherein each vent extends through the wall of the inner cannula body 210. Such vents permit any substance or compound delivered from the external source to enter into the lumen 221 of the internal cannula 200 before passing into the respiratory system of the patient.

Another aspect of the present invention includes a method of delivering an external compound or substance using the cannula system 100 in accordance with any embodiment disclosed within this specification or otherwise apparent from the descriptions herein. In embodiments, an outer cannula 300 is inserted into a stoma opening and in to the trachea of a patient. An internal cannula 200 can then be inserted within the lumen of the outer cannula 300. An external source can be secured to the port 313 of the outer cannula 300 and the external source can be engaged such that a substance or compound is introduced into the respiratory system of the patient.

In embodiments, the external source is attached to the port 313 via a Luer-lock hub or any other mechanism known to produce a reversible connection therebetween. In alternative embodiments, the external source is attached to the port 313 via a frictional connect, such as through the sliding of tubing over the tubular extension 312.

Also disclosed is a kit that includes a canula system 100 in accordance with any embodiment disclosed within this specification or otherwise apparent from the descriptions herein. In embodiments, the kit comprises an internal cannula 200, an outer cannula 300, and instructions for use or assembly of the cannula system 100. The instructions can be physically provided with the kit or accessible separately from the kit, such as via the retailer's or manufacturer's website.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

The disclosed technology centers around design and functional improvements of the cannula portion of tracheostomy and endotracheal tubes. Tracheostomy and endotracheal tubes are commonly used devices for creating or securing a temporary or permanent breathing passage. The commercial applications of tracheostomy and endotracheal tubes are well documented. The presently disclosed design improvements provide additional capabilities including oxygen and drug delivery capabilities. The design improves the utility, efficiency and capabilities of these indwelling cannulas while also reducing possible complications including mucous plugging.

These presently disclosed technology innovations help resolve existing problems with the use of tracheostomy and endotracheal tubes.

### Cannula Innovation for Tracheostomy and Endotracheal Tubes

### Humidification

1. Problem: Current cannula designs are usually provided using T-piece that drags on the tracheostomy due to the weight of the tubing/ventilator or via face mask placed near the tracheostomy which is inefficient.
2. Solution: Create a port on the side(s) of the outer tube of the tracheostomy above the flange that can deliver oxygen and humidified air through a channel between the outer and inner cannula. Small vents in the inner cannula may further allow this humidified oxygen to filter into the inner cannula as well. Lighter weight oxygen tubing can then be attached to these ports which will reduce drag on the tracheostomy tube and deliver humidified oxygen more effectively. Similarly, such a design for endotracheal tubes without the perforations on the inner cannula could be an alternative source for ventilation.
3. Additional advantages—by creating airflow and humidification within and around the inner cannula, reduces the likelihood of mucus plugging of the tracheostomy tube itself. This can be useful for both tracheostomy and endotracheal tubes.
4. Currently available oxygen adaptor (see FIG. 1)—Currently available adapters such as that depicted in FIG. 1 allow for direct flow of oxygen but a disadvantage is drag of the tubing on the tracheostomy that will create possibility of moving the position of the tube, dislodgement, and wounds resulting from the tracheostomy burying into the skin.
5. Currently available mask adaptor (see FIG. 2)—Currently available face-mask-type adapters such as that depicted in FIG. 2 provide inefficient delivery due to patient movement and constant need for adjustment. If you cannot use a tie around the neck (e.g. patients with head neck flaps), you then need to find a way to tape these face masks to the chest or neck using silk tape which is inefficient

### Drug Delivery

1. Problem: Current cannula designs do not include the ability to inject or provide aerosolized drugs without disconnecting the ventilator circuit
2. Solution: A port on the outer cannula or connecting to a drug delivery channel for tracheostomy and endotracheal tubes respectively will allow this function to be created in a patient needing ventilator support.
3. Advantages: This ability to deliver drugs without disconnecting the circuit prevents interruption of treatment, facilities care givers to management for patients and reduces repeated aerosolization of infective particles in particular infectious scenarios such as COVID 19 positive patients on a ventilator.

### Equivalents

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A respiratory cannula system comprising:
an outer cannula further comprising (1) an outer cannula body, (2) an outer cannula lumen, and (3) a port in the form of a hole, gap, notch, or channel extending through the outer cannula body;
an inner cannula further comprising an inner cannula body and an inner cannula lumen;
wherein the outer cannula is configured to receive and hold at least a portion of the inner cannula body within at least a portion of the outer cannula lumen; and
a chamber exists in the outer cannula lumen when the inner cannula is disposed therein, the chamber being in fluid communication with a patient's respiratory system.

2. The respiratory cannula system of claim 1, wherein the outer cannula lumen comprises a diameter that is slightly larger than a diameter of the inner cannula body; and the chamber exists as a space within the outer cannula lumen when at least a portion of the inner cannula body resides therein.

3. The respiratory cannula system of claim 2, wherein the diameter of the inner cannula body is about 80% of the diameter of the outer cannula lumen.

4. The respiratory cannula system of claim 2, wherein the outer cannula lumen comprises a diameter of about 10 mm or less.

5. The respiratory cannula system of claim 4, wherein the outer cannula lumen comprises a diameter of about 6 mm, about 7 mm, about 8 mm, about 9 mm, or about 10 mm.

6. The respiratory cannula system of claim 2, wherein the inner cannula body comprises a diameter of about 8.5 mm or less.

7. The respiratory cannula system of claim 6, wherein the inner cannula body comprises a diameter of about 4.5 mm, about 5.5 mm, about 6.5 mm, about 7.5 mm, or about 8.5 mm.

8. The respiratory cannula system of claim 2, wherein the outer cannula lumen comprises a diameter of about 10 mm or less and the inner cannula body comprise a diameter of about 8.5 mm or less.

9. The respiratory cannula system of claim 8, wherein the outer cannula lumen comprises a diameter of about 8 mm and inner canula body comprises a diameter of about 6.5 mm.

10. The respiratory cannula system of claim 1, wherein the port comprises a tubular extension extending from an external surface of the outer cannula body.

11. The respiratory cannula system of claim 1, wherein the port permits fluid communication between an external source and the chamber.

12. The respiratory cannula system of claim 11, wherein the port comprises a valve to selectively control fluid communication with the chamber.

13. The respiratory cannula system of claim 11 wherein the port further comprises a Luer-lock or Luer-taper hub.

14. The respiratory cannula system of claim 1, wherein the outer cannula comprises at least two ports.

15. The respiratory cannula system of claim 1, wherein the inner cannula body further comprises at least one vent that is configured to permit fluid communication between the inner cannula lumen and the chamber, the port, or a combination thereof

16. The respiratory cannula system of claim 15, wherein the at least one vent comprises a plurality of perforations disposed along the inner cannula body.

17. The respiratory cannula system of claim 11, wherein the external source is configured to introduce oxygen, humidified air, a pharmaceutical agent, or a combination thereof into the chamber.

18. The respiratory cannula system of claim 17, wherein the pharmaceutical agent comprises one or more aerosolized drugs.

19. The respiratory cannula system of claim 17, wherein the pharmaceutical agent comprises albuterol, dexamethasone, tobramycin, any other medication that can be delivered via a nebulizer, or a combination thereof

20. The respiratory cannula system of claim 1, wherein the outer cannula, inner cannula, or both comprise a medical grade metal, a medical-grade polymer, or a combination thereof

21. The respiratory cannula system of claim 20, wherein the medical grade metal comprises stainless steel, titanium, tantalum, gold, platinum, palladium, or a combination thereof

22. The respiratory cannula system of claim 20, wherein the outer cannula, inner cannula, or both comprise a silicone elastomer, sterilizable plastic, polytetrafluoroethylene, polyether block amide, polyvinyl chloride, or a combination thereof

23. The respiratory cannula system of claim 1, wherein the outer cannula, inner cannula, or both are reusable.

24. The respiratory cannula system of claim 1, wherein the outer cannula, inner cannula, or both are disposable.

25. A method of delivering humidified air to a patient with a tracheostomy, the method comprising:
obtaining the respiratory cannula system of any one of claims 1-24;
inserting the outer cannula through a stoma and into the trachea of a patient;
inserting the inner cannula into the outer cannula lumen;
attaching an external source of humidified air to the port of the outer cannula;
starting the flow of humidified air through the port, into the chamber, and into the respiratory system of the patient.

26. A method of delivering oxygen to a patient with a tracheostomy, the method comprising:
obtaining the respiratory cannula system of any one of claims 1-24;
inserting the outer cannula through a stoma and into the trachea of a patient;
inserting the inner cannula into the outer cannula lumen;
attaching an external source of oxygen to the port of the outer cannula;
starting the flow of oxygen through the port, into the chamber, and into the respiratory system of the patient.

27. A method of delivering a pharmaceutical agent to a patient with a tracheostomy, the method comprising:
obtaining the respiratory cannula system of any one of claims 1-24;
inserting the outer cannula through a stoma and into the trachea of a patient;
inserting the inner cannula into the outer cannula lumen;
attaching an external source of the pharmaceutical agent to the port of the outer cannula;
starting the flow of the pharmaceutical agent through the port, into the chamber, and into the respiratory system of the patient.

28. An outer cannula for use in a respiratory cannula system comprising:
an outer cannula body, an outer cannula lumen, and a port in the form of a hole, gap, notch, or channel extending through the outer cannula body;
the outer cannula being configured to receive and hold at least a portion of an inner cannula body within at least a portion of the outer cannula lumen;
the outer cannula lumen comprising a diameter that is slightly larger than a diameter of the inner cannula body such that a chamber exists within the outer cannula lumen when an inner cannula body resides therein;
the port being in fluid communication with the chamber; and
the chamber being in fluid communication with a patient's respiratory system.

29. The outer cannula of claim 28, wherein the port comprises a tubular extension extending from an external surface of the outer cannula body.

30. An inner cannula for use in a respiratory cannula system comprising:
an inner cannula body and an inner cannula lumen;
at least a portion of the inner cannula body being configured to reside within an outer cannula lumen comprising a diameter that is slightly larger than a diameter of the inner cannula body such that a chamber exists within the outer cannula lumen when at least a portion of the inner cannula body is disposed therein.

31. The inner cannula of claim 30, wherein the inner cannula body further comprises at least one vent that is configured to permit fluid communication between the inner cannula lumen and the chamber.

32. The inner cannula of claim 31, wherein the at least one vent comprises a plurality of perforations disposed along the inner cannula body.

33. A kit for use in a respiratory cannula system comprising:
an outer cannula further comprising (1) an outer cannula body, (2) an outer cannula lumen, and (3) a port in the form of a hole, gap, notch, or channel extending through a portion of the outer cannula body;
an inner cannula further comprising an inner cannula body and an inner cannula lumen;
wherein the outer cannula is configured to receive and hold at least a portion of the inner cannula body within at least a portion of the outer cannula lumen; and
a chamber exists in the outer cannula lumen when the inner cannula is disposed therein, the chamber being in fluid communication with a patient's respiratory system;
instructions for use of the kit.

34. The kit of claim 33 further comprising a pharmacological agent for distribution into the respiratory system of the patient through the port of the outer cannula.
